(19) United States
(12) Patent Application Publication
(10) Pub. No.: US 2018/0279937 A1
(43) Pub. Date: Oct. 4, 2018

(54) METHOD AND SYSTEM FOR DIAGNOSTIC PUPILLOMETRIC ASSESSMENT OF TRAUMATIC BRAIN INJURY

(71) Applicant: BAE Systems Information and Electronic Systems Intntegration Inc., Nashua, NH (US)

(72) Inventors: John Medberry, Austin, TX (US); Mark J. Dube, McDade, TX (US); Will R. Grigsby, Austin, TX (US); John D. Johnson, Thrall, TX (US); Joseph S. McGaughey, Austin, TX (US); Mitchell J. Nord, Round Rock, TX (US); Gaylan L. Brannan, Bastrop, TX (US); Somit S. Mathur, Austin, TX (US)

(21) Appl. No.: 15/765,935

(22) PCT Filed: Oct. 7, 2016
(86) PCT No.: PCT/US16/56003

§ 371 Date: Apr. 4, 2018

Related U.S. Application Data
(60) Provisional application No. 62/238,287, filed on Oct. 7, 2015.

(51) Int. Cl. A61B 5/00; A61B 3/14; A61B 3/11; A61B 3/00

(52) U.S. Cl. CPC A61B 5/4064; A61B 3/0008; A61B 3/112; A61B 3/145

(57) ABSTRACT

The system and method of size independent pupillometry used for assessing critical diagnostic characteristics relating to traumatic brain injury (TBI). The system and method utilizes the timing of a response to a stimuli for one or more pupils of an individual, characterizes the one or more timed responses, determines the latency of the one or more timed responses, measures the minimum constriction value for the one or more timed responses; and assesses the one or more timed responses collected to detect changes in neurotransmission and neuroactivity of an individual to assess the presence of a traumatic brain injury.

### CROSS REFERENCE TO RELATED APPLICATIONS

This Application claims the benefit of U.S. Provisional Patent Application No. 62/238,287, filed Oct. 7, 2015, the content of which is incorporated by reference herein in its entirety.

### FIELD OF THE DISCLOSURE

The present disclosure relates to pupillometry and more particularly to a pupil size agnostic method of pupillometry used for assessing critical diagnostic characteristics relating to traumatic brain injury.

### BACKGROUND OF THE DISCLOSURE

Healthcare providers currently use one or more tests to assess whether or not a person has suffered from a traumatic brain injury (“TBI”). Tests range from speech and language tests, cognition and neuropsychological tests, to various forms of imaging tests. Some imaging tests include computerized tomography (CT) scans, magnetic resonance imaging (MRI), and intracranial pressure monitoring (ICP). Additionally, the study of a person's ocular response to light may also be used. One such test is the “swinging light” test where a light source, such as a penlight or flashlight, is moved back and forth in front of a patient to see the eye's reaction to light. Typically, each pupil will constrict when a light is shined on the eye and the opposite pupil will constrict consensually.

When a person has suffered TBI, there is often damage to the optic nerve. This damage reduces the sensory (afferent) stimulus sent to the midbrain. With TBI, the pupil responds less vigorously and dilates more slowly from its prior constricted state when the light source is moved away from the unaffected eye towards an affected eye. This response is known as an afferent pupillary defect.

Existing measurements for pupillometry and the diagnostic characterizations related to it measure the linear or areal aspects of the pupil. Existing methods look at changes over time and apply threshold limits on those values. However, these products report multiple false detects due to the nature of the type of measurement taken. Current methods are also inherently tied to the size of the pupil in the evaluation and do not account for wide variations in pupil size within the population. A variation in pupil size greatly degrades the quality of the measure. Normalization does not exist in these conventional methods that also typically rely on baseline measurements to determine if there has been a change, which may not he available.

These conventional TBI methods examine the size of the pupil and use the timing of the responses, characteristics of the time response, latency, the minimum constriction value for TBI analysis and results in poor accuracy and false detects.

### SUMMARY OF THE DISCLOSURE

It has been recognized that current methods of pupillometry are prone to false detects and are inherently biased by the size of the pupil. The techniques of pupillometry of the present disclosure decouple pupil measurements from the size of the pupil and use the timing of the responses, characteristics of the time response, latency, the minimum constriction value, and the like to explicitly provide for a more accurate assessment with fewer false detects.

One aspect of the present disclosure is a pupil size agnostic method of pupillometry used for assessing critical diagnostic characteristics relating to neurotransmission and neuroactivity comprising, timing the response to a stimuli for one or more pupils of an individual, characterizing the one or more timed responses, determining the latency of the one or more timed responses measuring the minimum constriction value for the one or more timed responses; and assessing the one or more timed responses collected to diagnose changes in neurotransmission and neuroactivity for an individual.

Another aspect of the present disclosure is a pupil size independent method of pupillometry used for assessing traumatic brain injury in an individual comprising capturing one or more images of one or more pupils of an individual that are dark adapted; determining a pre-stimulus maximum diameter for the one or more pupils of an individual; providing a stimulus to one or more pupils of an individual; capturing one or more images of one or more pupils of an individual following the stimulus; timing the one or more pupils' response to the stimulus; measuring the extent of the one or more pupils' response to the stimulus; characterizing the one or more pupils' response to the stimulus; determining the onset of constriction for the one or more pupils; measuring the minimum constriction value for the one or more pupils; and analyzing the one or more pupils' responses to the stimulus to assess traumatic brain injury in an individual.

One embodiment of the method further comprises providing illumination to one or more pupils of an individual to allow for dark adaptation. An embodiment is wherein the illumination is provided by one or more IR LEDs for at least 8 s.

One embodiment is wherein the stimulus is provided by one or more visible LEDs for about 70 ms. Another embodiment is wherein the steps of capturing one or more images of one or more pupils of an individual is with one or more CCD or CMOS cameras.

An embodiment of the method is wherein determining the onset of constriction comprises determining the time at which the one or more pupils is 95% dilated. Another embodiment is wherein analyzing the one or more pupils' responses to the stimulus comprises comparing a papillary response in a first eye of an individual to the pupillary response in a second eye of an individual.

One embodiment further comprises assessing the relationship between the pre-stimulus maximum diameter and the minimum constriction value for the one or more pupils to assess TBI in an individual. One embodiment further comprises normalizing the data.

Another aspect of the present disclosure is a system for assessing traumatic brain injury in an individual comprising, a housing having a subject side and an operator side comprising; one or more eyepieces optically connected to one or more cameras; a lighting module configured to control one or more LEDS or other light sources; an imaging module configured to control one or more imaging devices; a power supply; a memory for storing and retrieving one or more images of one or more pupils of an individual as captured by the camera module; a system manager module configures to control the lighting module and the imaging module; and a processing module for assessing traumatic brain injury in an individual.

One embodiment of the system further comprises a display or other user interface. An embodiment of the system is wherein the one or more LEDs comprises illuminating and stimulating LEDS. In some embodiments of the system the illuminating LEDs are IR LEDs and the stimulating LEDs are visible LEDs.

Another embodiment of the system is wherein Me system manager module fluffier comprises rules for controlling the one or more LEDs or the one or more cameras. One embodiment of the system is wherein the processing module assesses traumatic brain injury in an individual by analyzing one or more images of one or more pupils of an individual. The images can be processed concurrently with the system on the site of the testing; the images can also be processed elsewhere or at another time.

These aspects of the disclosure are not meant to be exclusive and other features, aspects, and advantages of the present disclosure will be readily apparent to those of ordinary skill in the art when read in conjunction with the following description, appended claims, and accompanying drawings.

### BRIEF DESCRIPTION OF THE DRAWINGS

The foregoing and other objects, features, and advantages of the disclosure will be apparent from the following description of particular embodiments of the disclosure, as illustrated in the accompanying drawings in which like reference characters refer to the same parts throughout the different views. The drawings are not necessarily to scale, emphasis instead being placed upon illustrating the principles of the disclosure.

FIG. 1A shows the variation between the left and the right eye for the same individual for one embodiment of the present disclosure.

FIG. 1B shows normalized data, smoothed and scaled to pre-flash value for one embodiment of the present disclosure.

FIG. 2A shows the correlation of latency and pupil diameter for one embodiment of the present disclosure.

FIG. 2B shows a distribution plot of constriction latency for the left eye and for the right eye according to one embodiment of the present disclosure.

FIG. 2C shows a distribution plot of pupil diameter for the left eye and for the right eye according to one embodiment of the present disclosure.

FIG. 3A shows a correlation between the minimum pupil diameter and the time of the minimum value according to one embodiment of the present disclosure.

FIG. 3B shows a distribution plot of the time of the minimum value for the left eye and for the right eye according to one embodiment of the present disclosure.

FIG. 3C shows a distribution plot of minimum pupil diameter for the left eye and for the right eye according to one embodiment of the present disclosure.

FIG. 4 shows a correlation of the minimum and the maximum values of the extent for one embodiment of the present disclosure.

FIG. 5A shows a traumatic brain injury response and the relative latency between eyes of an individual for one embodiment of the present disclosure.

FIG. 5B shows a traumatic brain injury response and the relative time of the minimum for one embodiment of the present disclosure.

FIG. 6A shows a plot of pupil diameter % over time with flash occurring in left eye of an individual for one embodiment of the present disclosure.

FIG. 6B shows a plot of pupil diameter % over time with flash occurring in the right eye of an individual for one embodiment of the present disclosure.

FIG. 6C shows a plot of pupil diameter % over time with flash occurring in left eye of an individual with suspected traumatic brain injury (TBI) for one embodiment of the present disclosure.

FIG. 6D shows a plot of pupil diameter % over time with flash occurring in the right eye of an individual with suspected traumatic brain injury (TBI) for one embodiment of the present disclosure.

FIG. 7 shows raw diameter data (mm) plotted over time (seconds) for one embodiment of the present disclosure.

FIG. 8 shows raw diameter data (mm) scaled to pre-flash mean and the minimum is scaled to zero for one embodiment of the present disclosure.

FIG. 9 shows high consistency in the time duration from pre-flash values to minimum values for diameter data according to the principles of one embodiment of the present disclosure.

FIG. 10 shows a screenshot of one embodiment of the system according to the principles of the present disclosure.

FIG. 11 shows one embodiment of the system of the present disclosure.

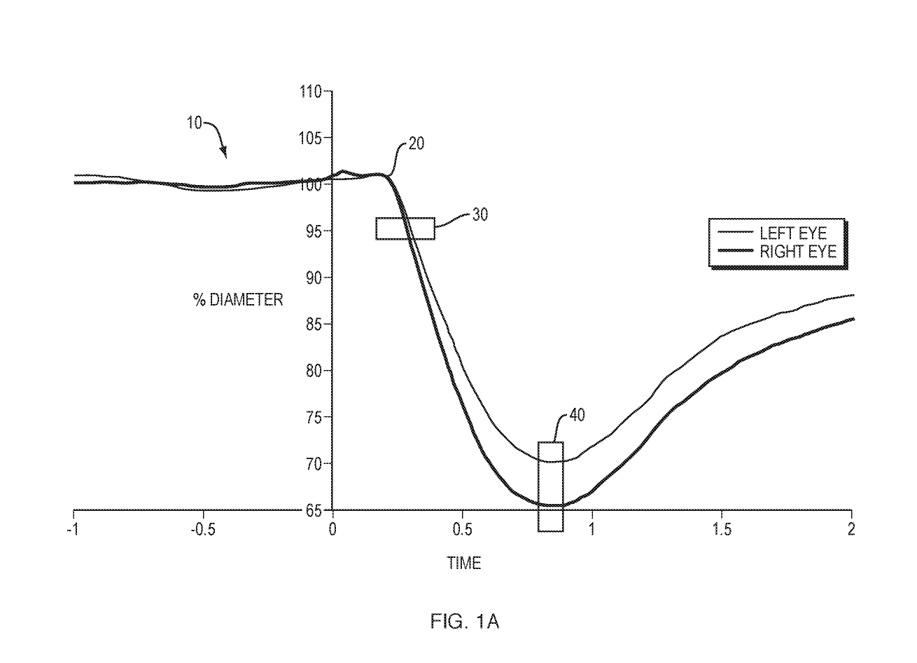

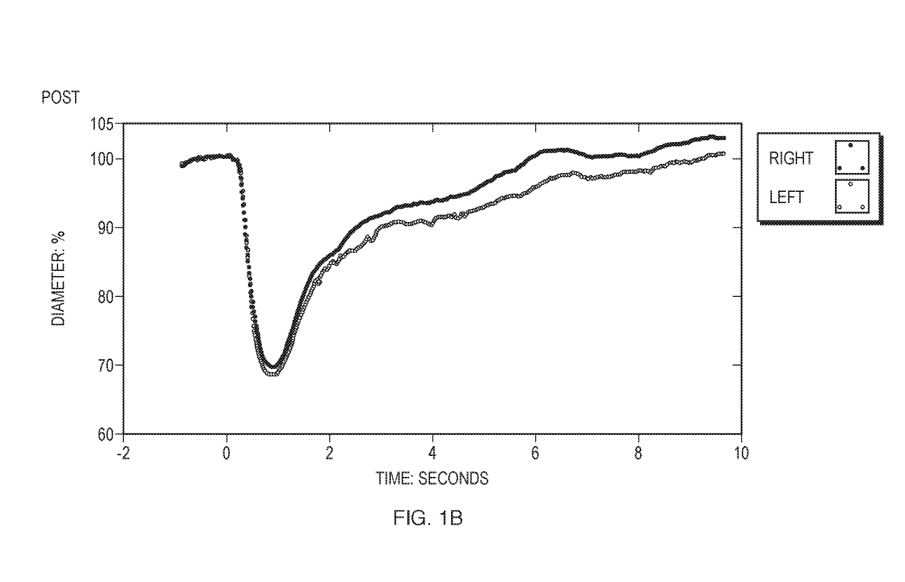

FIG. 1.2 shows a flow chart of one embodiment of the system of the present disclosure.

FIG. 13 shows one embodiment of the system of the present disclosure.

FIG. 14 shows a flow chart of the processing for one embodiment of the system of the present disclosure.

### DETAILED DESCRIPTION OF THE DISCLOSURE

Existing measurements for pupillometry and the diagnostic characterizations related to it measure the linear or areal aspects of the pupil as a function of time and apply threshold limits on those values. Current methods typically rely on a pupil measurement and a baseline pass/fail criteria based upon a threshold of the time rate of change of the pupil extent. This inherently carries the size of the pupil in the evaluation. The variation in pupil size greatly degrades the quality of the measure. In contrast, methods of pupillometry of the present disclosure decouple measurements of the size of the pupil and use the timing of the responses, characteristics of the time response, latency, the minimum constriction value, and the like to explicitly provide a more accurate assessment. In certain embodiments, the S statistical improvement (tightening of the distribution) of the method of the present disclosure compared to other implemented methods is about an order of magnitude improvement.

Current methods utilize the time rate of change of the pupil extent, and are highly dependent of pupil variation in the population. Because of this, current methods have a wide scatter of responses and generate many false detects on healthy individuals. The variation spills well outside of the current threshold limits.

In contrast, certain embodiments of the present disclosure have at least two measurements which are much more tightly linked to the physiological response. Thus, the incidence of false failure drops by about an order of magnitude or more by characterizing nerve signal transport time and neuro-muscular action. In certain embodiments, the measure is very consistent in healthy individuals.

Generally, a pupilometer measures the mean pre-stimulus value of the pupil and then the pupil is measured over time for the duration of the assessment. A review of previously collected data showed considerable variation in pupil size within the human population. It was also clear that with the range of resultant constriction, a viable, consistent measure was not determinable using current methods. For example, certain state of the art products reported false detections a great deal of the time (e.g., reported that a person had been exposed to neurotoxin when in fact they had not been). For traumatic brain injury patients, the present disclosure provides guidance and avoids linear measurements resulting from pupil size variability. In certain embodiments, the present techniques are divorced from the extent measurement of the pupil. In certain examples, this is done using feature extraction from the pupil extent versus time to provide a consistent measure with lower variability. In one embodiment of the present disclosure, feature extraction on the onset of response (e.g., constriction) and at the minimum extent (i.e., at its most constricted) provides a consistent measure for this particular set of physiological responses. By extracting features in this way, dependency on the extent of the pupil is removed and gives a more reliable metric for assessment.

One aspect of the present disclosure is a population independent parameter for pupil response evaluation. The present method is of high intrinsic value due to its consistency and it provides an assessment of neurological related response changes that allow for very high confidence assessment with very low false failures. It is understood that existing methods do not provide the robust level of detection or the same level of consistency as described herein.

Current methods of metric based pupillometry rely intrinsically on pupil extent and the evaluation of condition states of the subject based upon the extent measurement (e.g., diameter, radius, area) and the extent measurement per unit time (e.g., rate). Presented here, along with validation, is a method of measurement that is independent of biometric variation in the normal population. The method of the present disclosure provides a consistent assessment tool for evaluation of critical diagnostic characteristics related to the field of pupillometry. More specifically, the method relates to assessing neurotransmission and neuroactivity in a pupil size independent manner.

Pupillometry is used in subject assessment for a host of potential negative states; these include, but are not limited to, exposure to neuro-active toxins, traumatic brain injury, and basic health states such as hypertension, multiple sclerosis, and the like.

Existing methods establish the measurement of the pupillary response based on a measurement of pupil motility (time rate of change of the diameter, area or radius), measurement of the constricted and dilated pupil, and other similar methods. All of the values inherently rely on the pupil of the individual, and these responses are compared to a prior established rate. However, the inclusion of the extent measurement requires inclusion of the subject-dependent measure of the pupil. Even self-rectified values (e.g., using percent) include an inherent linear extent. Further, basing the measure on the extent of the pupil constrains two otherwise independent biometric measures into one which further makes for a biased measurement.

The basic characteristics involved in the extent measurement are the unstimulated maximal pupil extent and the amount of available constriction. The maximal pupil extent has a wide range of variation within the population. The amount of available constriction is coupled to both the population variation and the degradation of motility over the lifetime of an individual. A measurement that is consistent removes both of these variations from the evaluation and still provides useful diagnostic information.

The method presented herein relies on the basic underlying mechanisms in the pupillary response. The governing response to a stimulus of light is dictated by one set of muscles to constrict the pupil. The governing response in the absence of this stimulus is a counter set of muscles to widen the opening of the pupil. In certain embodiments, the independent characteristics of these two sets of responses are used for population independent measurements along with the precise timing of the action of these responses.

In the absence of background visible light, the pupil opens to an asymptotic wide clear aperture. As this change in pupil size is governed by the weaker dilation muscle group, measurement of this asymptote is generally convolved with other effects within the individual. For example, measurement of a time spread mean within the point over time (e.g., measure of the extent averaged over time) provides a self-referenced comparative, base value. Application of a stimulus, e.g., a visible light source, for a brief period, activates the constriction. (miosis) driven by the constriction muscle group. Measurement of the extent over time (at each time sample) is required. In one embodiment of the present disclosure, the time series of the measurement of the extent is used as a point from which to extract independent values. The onset of constriction, or the constriction latency, is one of such independent values.

In one embodiment of the present disclosure, regardless of the clear aperture base value, the time for latency was consistent within the unaffected population evaluated. In one embodiment, a second parameter that is independent of the population is the time of the minimum compression. These are time dependent values which are independent of the sample. These values are specifically related to neurotransmission. and neuroaction values, regardless of pupil size.

According to certain testing, multiple individuals were assessed utilizing the following method: for all times an image was acquired of the eye; eyes were dark adapted, illuminated only by IR LEDs (e.g., NIR about 850 nm) for 8 seconds; and at 8 seconds, a 70 ms visible light flash from a white LED was applied to one eye. In certain embodiments, the system incorporated dual eye measurement and the data included both eyes. Data was collected for 10 additional seconds at a sampling rate of about 80 Hz. In certain embodiments, the pixel resolution for the apparatus was about 0.08 mm/pixel. The pupil extent was measured from the imagery. In one example, the diameter was in millimeters. The response of the diameter of the pupil was plotted as a function of time. In one embodiment, the diameter versus time data was processed to measure the constriction latency and the time to minimum value. In certain embodiments, the point in the data at which the dimeter was 95% of the maximum value was measured from the time series as the onset of constriction. The time of the minimum value was also extracted from the time series. In certain embodiments, a fitting function was used for smoothness in the minimum.

Referring to FIG. 1A and 1B, images of a left and a right eye response for a single individual is shown and it illustrates the differing responses observed by % change in pupil dimeter over time. If this data was processed using previous methods, each eye would have generated a different slope due to the different depths of response. This illustrates that the response is eye dependent if the extent measurement is included. For traumatic brain injury, this illustrates that the only viable assessment route should use the time differential between the features for accurate assessment.

Referring to FIG. 1A, plots of a left and a right eye responding to a stimulus are shown. The x-axis represents time (in seconds), with time 0 representing the visible flash. The flash occurs after 8 seconds of dark adaptation (IR LED exposure). In certain embodiments, the IR LEDs are on continuously. In certain embodiments, the IR LEDs have a radiant intensity of about 20 mW/sr. In certain embodiments, the IR LEDS are always on. The y-axis represents the % of pupil diameter, with 100% representing the pupil diameter prior to an applied stimulus 10. In certain embodiments, the 100% value represents the diameter of the pupil after dark adaption for about 8 seconds. In certain embodiments, the applied stimulus is a 70 ms flash of visible light from visible LEDs. In certain embodiments, the dark adaption duration and flash duration can be varied. In certain embodiments, the visible LEDs have a luminous intensity (lν) of about 2063 cd. In certain embodiments, the wavelength of the visible LEDs is about 850 nm. In certain embodiments, 20 represents the inflection point following the flash. In certain embodiments, the onset of constriction 30 is taken at 95% diameter. In certain embodiments, variation between a left eye's and a right eye's response to a stimulus 40 is present to some degree. Ideally, there wouldn't be any variation, because the consensual response is perfect. But under normal conditions, people don't have identical responses. However, when a person is exposed to a TBI, the normalized curves vary dramatically.

Referring to FIG. 1B, normalized data, smoothed and scaled to pre-flash value is shown. More specifically, both left and right eyes were sampled, and the plots illustrate a consensual response because both pupillary responses track each other with minimum variation. This was normalized to the pre-flash value and measurement was in linear extent units (%). In this example, both the left and the right eyes track in a consensual manner, which is indicative of normal population not exposed to TBI. In one example, the duration of test is 18 s, with a total of (18 s*80 samples/sec)=1440 samples (or images) per eye.

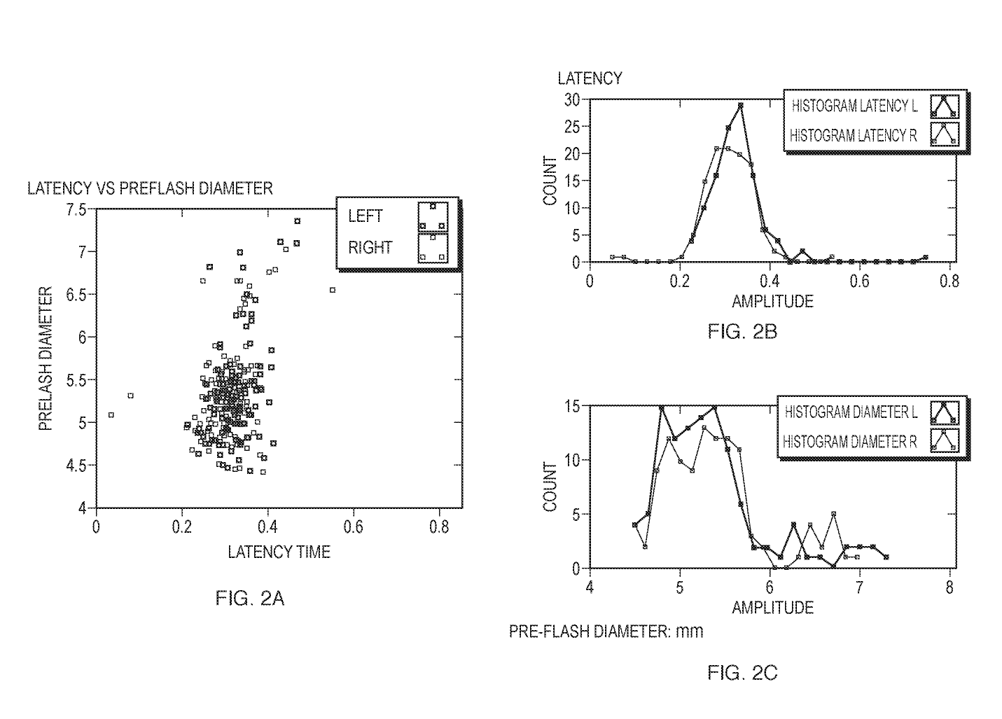

Referring to FIGS. 2A-2C, evaluation of the constriction latency, or onset of constriction, and evaluation of the correlation to the pupil diameter are shown for each eye. Review of the data, from a statistical viewpoint, shows significant independence of the latency time from the diameter. Referring to FIG. 2A, no specific correlation between the latency time and the pre-flash pupil diameter is found. Referring to FIG. 2B and FIG. 2C, the amplitude of the latency response shows a very Gaussian type (normal) distribution, and the spread of the pupil diameter within the population shows great variation, respectively.

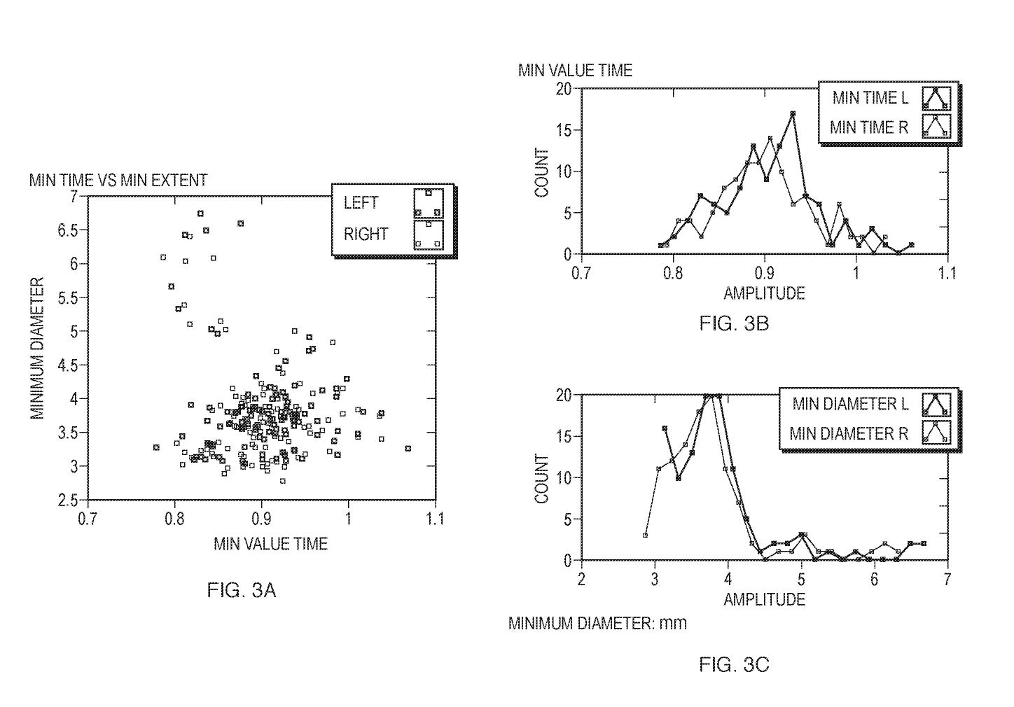

Referring to FIGS. 3A-3C, evaluation of the minimum extent and the time at minimum and the related correlations are shown. in FIG. 3A, the minimum value (i.e., smallest diameter) and the time of the minimum value is shown to be de-correlated from each other. On FIGS. 3B and 3C, the latency response histogram shows a very Gaussian type (normal) distribution, and the spread of the diameter is indicative of the population variation.

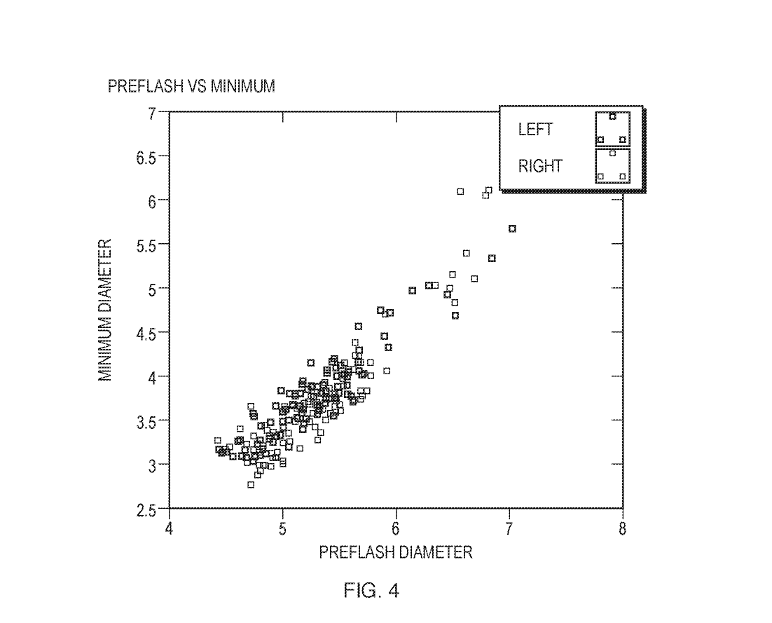

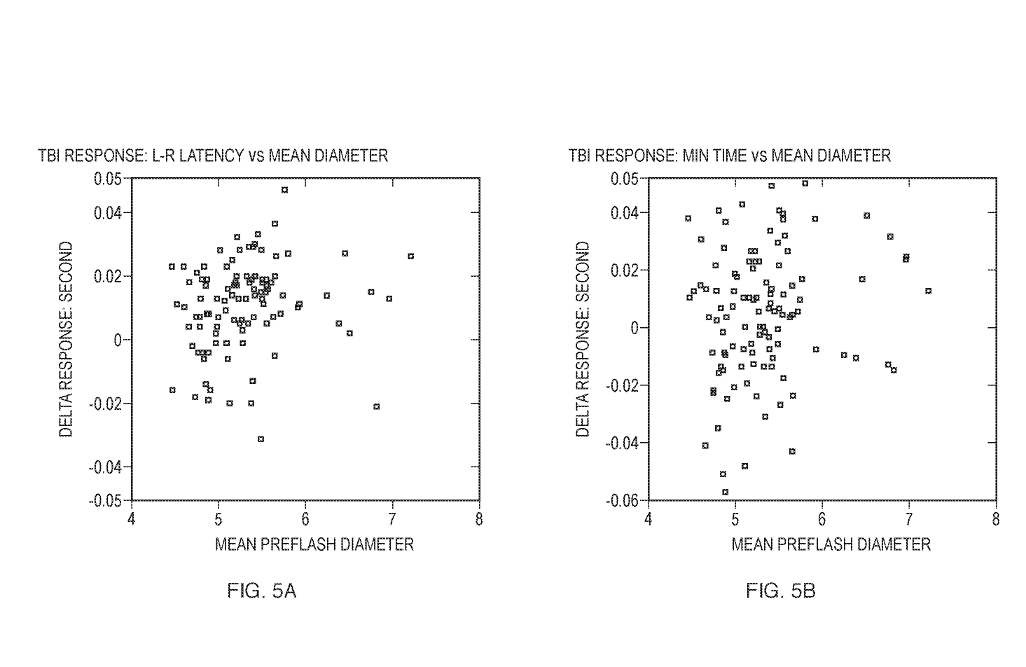

Referring to FIG. 4, correlation of the minimum diameter values and the maximum (pre-flash) diameter values of the pupil extent are shown. More specifically, the measurement of the rate of change is shown to be intrinsically related to the maximum value and the minimum value. FIG. 4 shows the correlation is strongly linear between pre-flash mean (e.g., widest diameter) and the minimum value attained (e.g., following the flash). In certain embodiments, the linear correlation of the maximum and the minimum is exploited, where a deviation from this trend is another characteristic that is independent of the variation in pupil size in a given population. Thus, a lack of linearity may be indicative of TBI.

Referring to FIGS. 5A and 5B, traumatic brain injury (TBI) responses are shown as relative latency between the left and right eyes of a single individual according to the principles of one embodiment of the present disclosure. As related to traumatic brain injury, the same two parameters are even more tightly correlated. The TBI response is a breakdown of the consensual response inhibiting or degrading the response of the unstimulated eye. Using the constriction latency (i.e., onset of constriction) and the time when the minimum diameter is reached for the two eyes of an individual provides for an assessment of TBI with no correlation to the size of the pupil involved. Referring to FIG. 5A, the spread between the latency responses of the two eyes of a single individual is shown, with no correlation to the diameter of the pupil. Referring to FIG. 5B, the differences in the time that the minimum value was reached for two eyes is shown, with no correlation to the diameter of the pupil. The high degree of consistent timing is illustrative of the physical processes being tied to identical transport phenomena. In one embodiment, deviation from this relationship will guide the assessment in traumatic brain injuries.

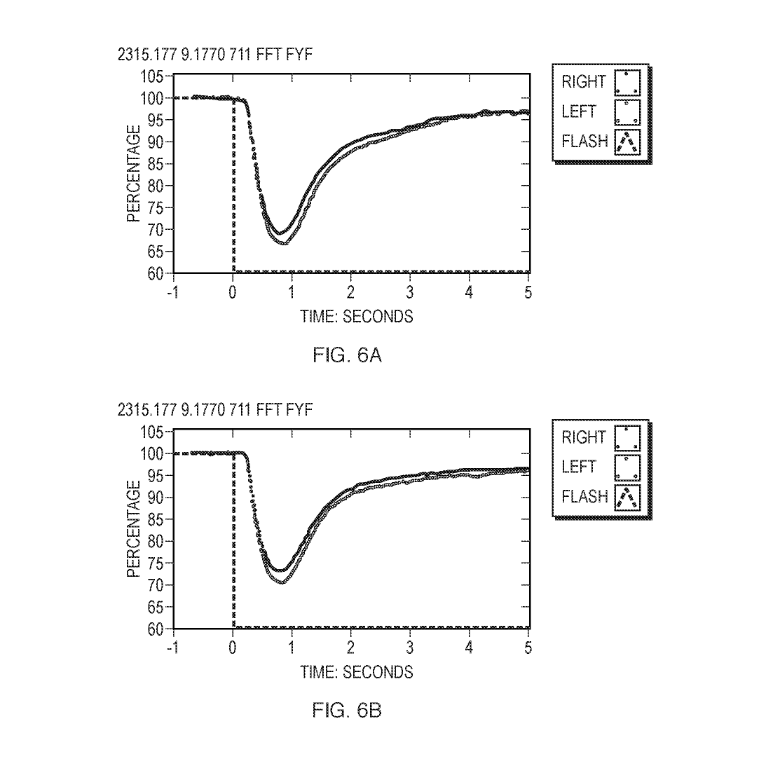

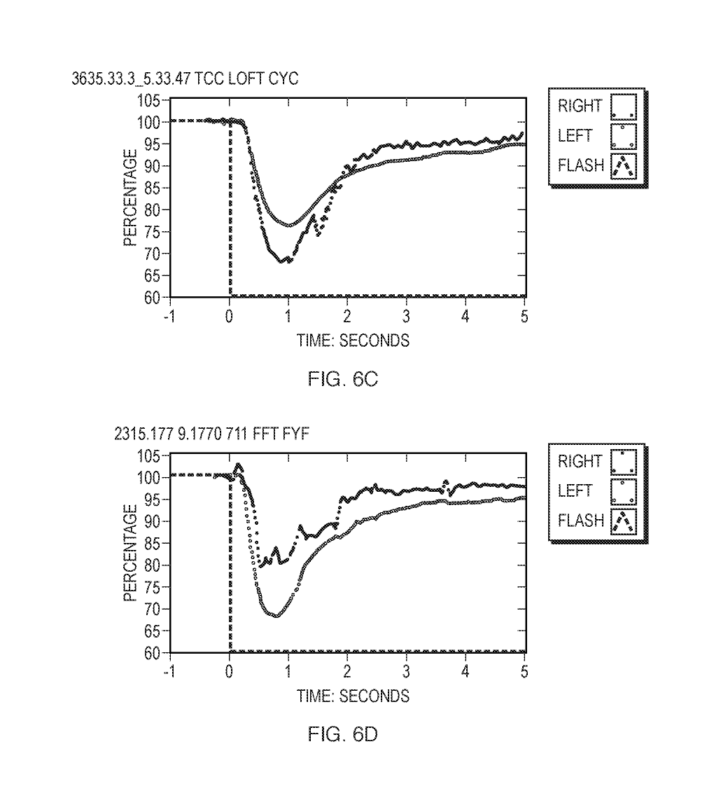

Referring to FIGS. 6A and 6B, plots of pupil size over time are shown for an individual according to one embodiment of the present disclosure. The flash occurs at time zero. In FIG. 6A, the flash is in the left eye and the right eye shows a consensual response. In FIG. 6B, the flash is in the right eye and the left eye shows a consensual response.

Referring to FIGS. 6C and 6D, plots of pupil size over time are shown for an individual known to have a traumatic brain injury (TBI) according to one embodiment of the present disclosure. The flash occurs at time zero. In FIG. 6C, the flash is in the left eye and the right eye shows a consensual response. In FIG. 6D, the flash is in the right eye and the left eye shows a consensual response. In comparing FIGS. 6C/6D to 6A/6B the individual with TBI has traces which are more dissimilar when compared to those without TBI. The departure from a consensual relationship is indicative of a TBI assessment. Moreover, this observation is a quantitative example of what physicians qualitatively observe when applying the “swinging light” test to patients with potential TBI. It may be possible to determine additional aspects of the extent of injury based on differences in smoothness of a trace.

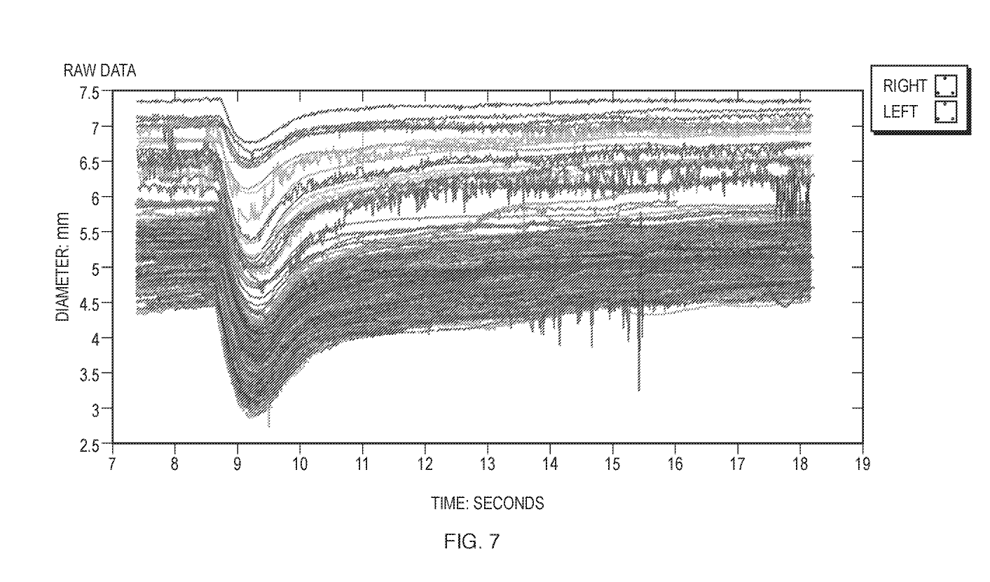

Referring to FIG. 7, raw diameter data is shown. More specifically, the vertical axis diameter is in millimeters and the horizontal is time in seconds. As can be seen, the consistency of the data, regardless of the measured diameter is very prevalent, with the response, in time, characteristic on every trace. In certain embodiments, initial diameter (after dark adaption) varies within the population and the data must be normalized. In certain embodiments, the slope to the minimum extent varies within the population as well.

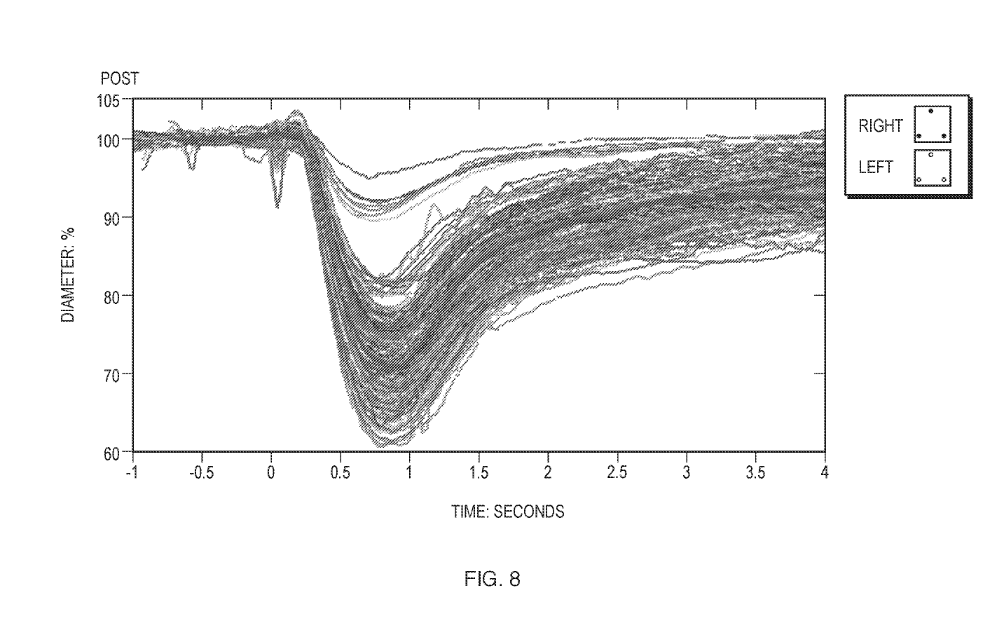

Referring to FIG. 8, raw data resealed with the minimum at zero and pre-flash mean at 1 is shown. The initial step in post processing is normalization to the pre-stimulus extent. Data shown in FIG. 8 used a 0.5 second duration mean divided through all other extent values. The range of slopes to the minimum is distinct.

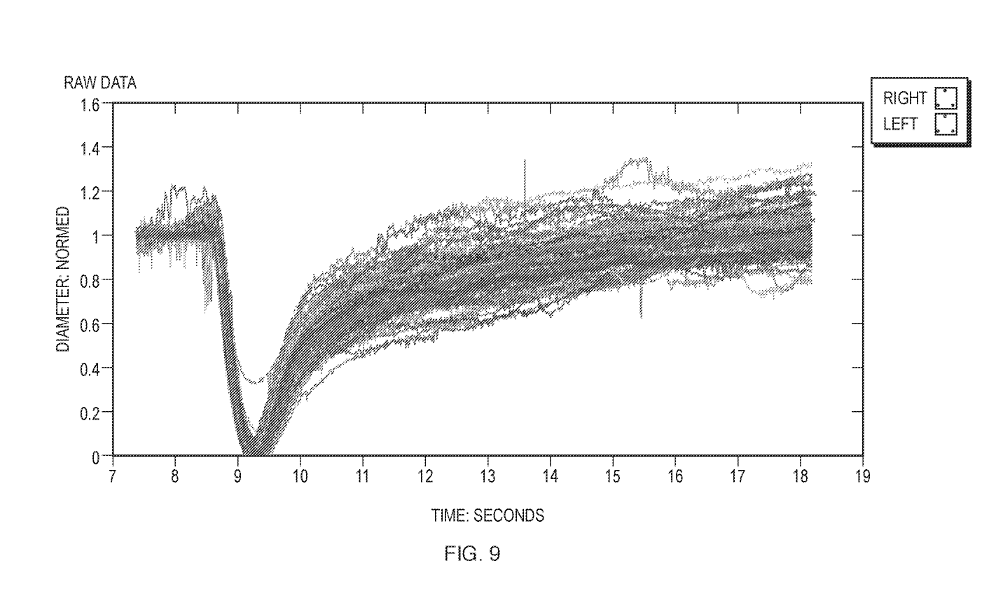

Review of the data presented in FIG. 9 shows high consistency in the time duration from pre-flash values to a minimum value. This is a function of the previously stated independence of the response on base extent.

From evaluation of the data presented so far, one question remained, ‘is there an equally independent evaluation possible for dilation?’ in certain embodiments, it is possible to segment pupil dilation/constriction into two time domain governed responses—an immediate impulse response followed by a minimum pupil diameter and then weak recovery. In certain embodiments, there is diversity in the time domain and amplitude of the recovery and it is inherently more condition dependent. The general trend appears to be a hand off between musculature responses, essentially from the cessation of the application of the constriction muscle group to the start of control by the dilation muscle group.

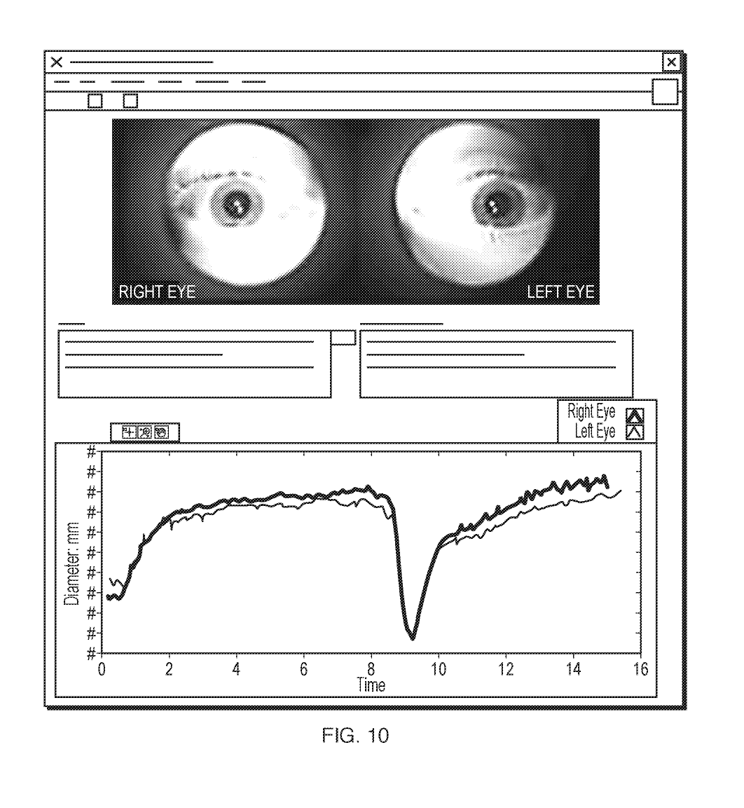

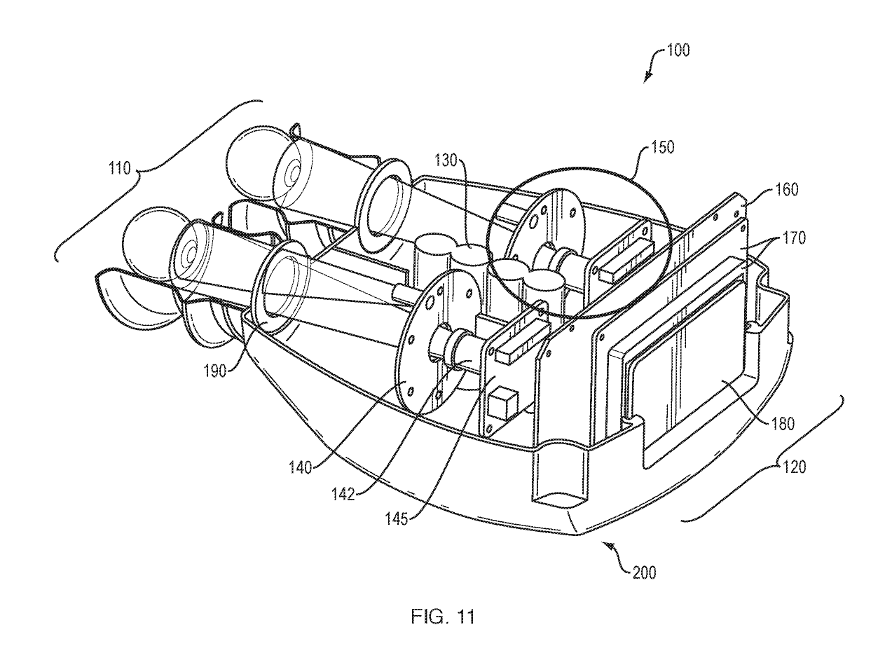

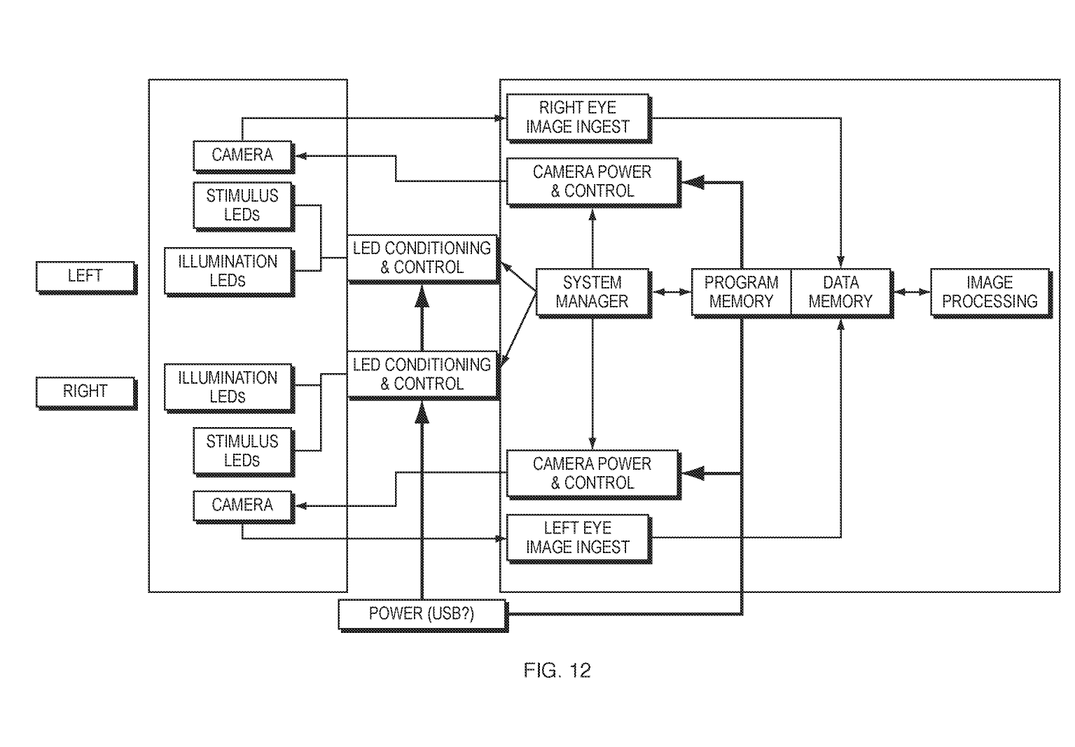

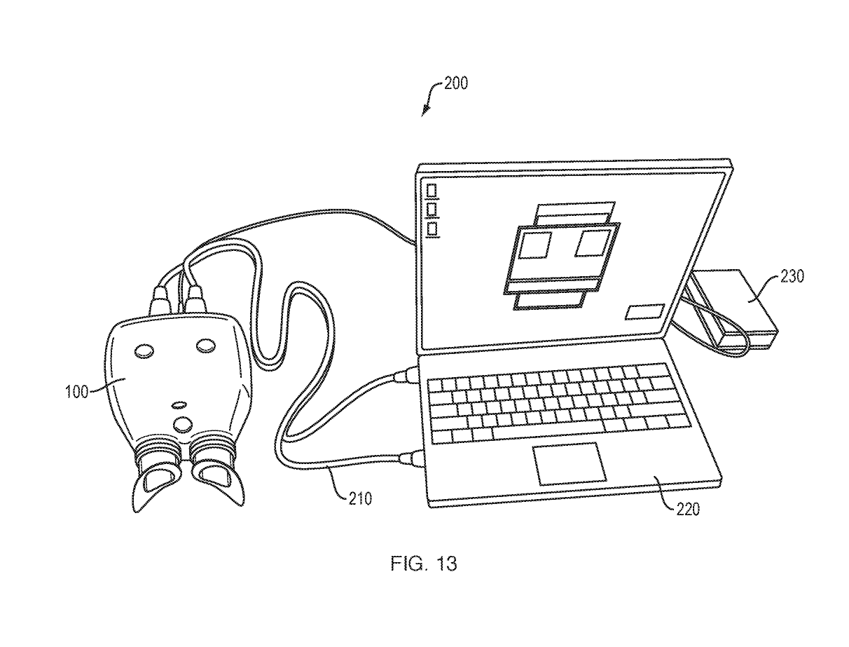

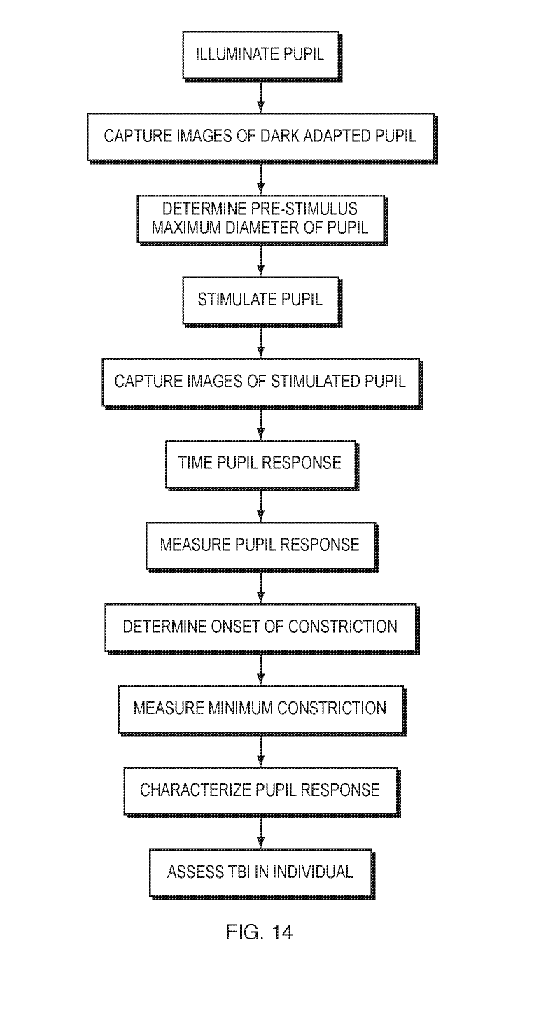

Referring to FIG. 10, a screenshot of one embodiment of the assessment system according to the principles of the present disclosure is shown. More particularly, the system comprises a graphical user interface (GUI) where analysis of the data can be viewed and assessed. In certain embodiments, the GUI is viewable on a portable device, such as a smart phone. In another embodiment, the GUI is resident on a laptop computer that is connected to the system via USB interface. There are several features which make the present approach unique and effective: 1) the system simultaneously captures ocular imagery from both eyes, enabling a quantitative assessment of consensual response; 2) the blockage of ambient light from both eyes isolates the consensual response to the stimulus created by the visible light flash in the system; and 3) the normalization of both measurements creates pupillary signatures that improve quality of measurement and statistical relevance.

In certain embodiments, following a period of dark adaptation, a visible flash is introduced to the left eye. In certain embodiments, following a period of dark adaptation, a visible flash is introduced to the right eye. In certain embodiments, following a period of dark adaptation, a simulated swinging light test is performed where a light flash alternates from the left to the right eye and back again. In certain embodiments, data is collected from the eye receiving the flash. In certain embodiments, data is collected from the eye that is responding consensually. In certain embodiments of the method of the present disclosure, the data is collected at a frequency of about 80 Hz and the data is normalized for later analysis.

Referring to FIG. 11, one embodiment of the system of the present disclosure is shown. More particularly, the system is comprised of a binocular device 100 having a subject side 110 and an operator side 120. In certain embodiments, the device is configured to view the response of pupils in both eyes upon stimulation of one or the other eye. In one example, the device is configured to stimulate (e.g., flash visible light) both eyes. The device may be contained in weather proof housing 200 in order to handle the external environment. In this example, the system has an onboard power supply, such as batteries 130 but can also be powered by external connection to a power source.

The device has light sources 140 to stimulate one or both eyes. In certain embodiments, the light sources can be IR LEDs or visible LEDs. The device has image detectors for each eye 145. In certain embodiments, the device has optical windows or screening to protect the detector lenses from physical damage 190. The device in this example has optics 142 in front of the imager to improve zoom and focus. The system can be coupled to a processing unit that employs a computer program to process the data. The device in a further example can transmit the data from the detector via wireless technology to a processing unit that is no co-located with the system. There is a Circuit Card Assembly (CCA) in this example that contains digital electronics 160 for capturing, storing and processing the data within the system. A modular imaging unit or camera assembly 150 is used to capture the images of the pupil during operation. On the operator side, this embodiment has a display 180 such as a liquid crystal display (LCD) with associated printed circuit board 170 to show the results of the processing to a user.

Referring to FIG. 12, a perspective view according to one embodiment of the system is shown. A binocular device is deployed proximate the face of the user to capture information from one or more eyes. In one example the system stimulates one eye while in another embodiment the system stimulates both eyes. There are one or more illumination light sources such as LEDs or lasers for each eye to provide the stimulation. These illumination. LEDs can be in the NIR range and provide a simulated darkness while still allowing for image capture of the eye at rest. ln certain embodiments, the system has one or more stimulus LEDs for each eye. In a further embodiment the stimulus LEDs are in the visible range and provide a “white” flash.

Still referring to FIG. 12, in certain embodiments the system has a camera for each eye such as CCD or CMOS cameras. In this example the device has LED modules for controlling the illumination LED brightness and duration. For example, the LED modules control the visible LED brightness and duration of flash. The LED control modules can be modifiable by the user. In one embodiment, a set of rules is present such that the device has a set protocol for controlling the LEDs. In other embodiments, there is a system manager for controlling the one or more LEDs and the one or more cameras. In other embodiments, the device has camera modules for controlling the one or more cameras and can be modifiable by the user. The device can also have power modules for controlling the power of the device. In certain embodiments, the power is USB. In certain embodiments, a set of rules is present such that the device has a set protocol for controlling the one or more LEDs and/or the one or more cameras.

Still referring to FIG. 12, in certain embodiments, the system has program memory. In certain embodiments, the system has data memory. In certain embodiments, the device process images on-board. In certain embodiments, the system has an embedded digital processor.

Using the method of the present disclosure requires some way to maintain the position of the subject's eyes relative to the device's image detector or compensate for variations in position. This may be accomplished several ways, either through a mechanism (e.g., a human head to device interface), optical design, or via an algorithm (e.g., measuring and correlating any change in size to the subject's facial features which do not change as rapidly as the pupil, such as the iris diameter or the distance from the inner and outer corner of the eye). Other methods may include a sonar sensor, sterographic imaging and/or triangulation, a 3D image sensor with depth of field, variable focus and/or zoom optics, or the like.

FIG. 13 shows an embodiment of the system of the present disclosure. This embodiment of the system has acquisition hardware 230 in communication with a laptop or other acquisition computing device 220. The system also has a binocular device 100 for use with an individual. In this example, the binocular acquisition device is connected via cables 201 to the acquisition computing device 220. In some systems, the connection is wireless. In other systems, a separate computing system is sued for analyzing the data and assessing TBI in an individual. In some cases the analysis is completed remote from acquisition.

FIG. 14 is a flow chart for the processing according to one embodiment. Here, one or more pupils are illuminated and become dark adapted. Then, images are captured of the one or more pupils. A pre-stimulus maximum diameter for the one or more pupils is determined. A stimulus is applied to the one or more pupils and images are collected of the stimulated pupil over a period of time. Over time, the one or more pupils will return to a dark adapted state if illuminated, as above. The timing of each pupil's response is determined as well as the minimum extent of the constriction for each pupil. The pupils are compared to each other to look for deviance from sympathetic responses as well as other characteristics discussed herein. The pupil's response is categorized and an assessment is completed to determine if an individual has experienced TBI.

While the principles of the disclosure have been described herein, it is to be understood by those skilled in the art that this description is made only by way of example and not as a limitation as to the scope of the disclosure. Other embodiments are contemplated within the scope of the present disclosure in addition to the exemplary embodiments shown and described herein. Modifications and substitutions by one of ordinary skill in the art are considered to be within the scope of the present disclosure.

What is claimed:

1. A pupil size independent method of pupillometry used for assessing traumatic brain injury in an individual comprising,
capturing one or more images of one or more pupils of an individual that are dark adapted;
determining a pre-stimulus maximum diameter for the one or more pupils of an individual;
providing a stimulus to one or more pupils of an individual;
capturing one or more images of one or more pupils of an individual following the stimulus;
timing the one or more pupils' response to the stimulus;
measuring the extent of the one or more pupils' response to the stimulus;
determining the onset of constriction for the one or more pupils;
measuring the minimum constriction value for the one or more pupils;
characterizing the one or more pupils response to the stimulus; and
analyzing the one or more pupils' responses to the stimulus to assess traumatic brain injury in an individual, wherein the one or more pupils' responses are pupil size independent.

2. The method of claim 1, further comprising providing illumination to one or more pupils of an individual to allow for dark adaptation.

3. The method of claim 2, wherein the illumination is provided by one or more IR LEDs for at least 8 s.

4. The method of claim 1, wherein the stimulus is provided by one or more visible LEDs for about 70 ms.

5. The method of claim 1, wherein the steps of capturing one or more images of one or more pupils of an individual is with one or more CCD or CMOS cameras.

6. The method of claim 1, wherein determining the onset of constriction comprises determining the time at which the one or more pupils is 95% dilated.

7. The method of claim 1, wherein analyzing the one or more pupils' responses to the stimulus comprises comparing a pupillary response in a first eye of an individual to the pupillary response in a second eye of an individual.

8. The method of claim 1, further comprising assessing the relationship between the pre-stimulus maximum diameter and the minimum constriction value for the one or more pupils to assess TBI in an individual.

9. The method of claim 1, further comprising normalizing the data.

10. An system for assessing traumatic brain injury in an individual comprising, a housing having a subject side and an operator side comprising;
one or more eyepieces optically connected to one or more imaging devices;
a lighting module configured to control one or more light sources;
an imaging module configured to control one or more imaging devices;
a memory for storing and retrieving one or more images of one or more pupils of an individual as captured by the imaging module;
a system manager module configured to control the light module and the imaging module; and
a processing module for assessing traumatic brain injury in an individual.

11. The system of claim 10, further comprising a display coupled to the housing.

12. The system of claim 10, wherein the one or more light sources comprises illuminating and stimulating LEDS.

13. The system of claim 12, wherein the illuminating LEDs are IR LEDs and the stimulating LEDs are visible LEDs.

14. The system of claim 10, wherein the system manager module further comprises rules for controlling the one or more light sources or the one or more imaging devices.

15. The system of claim 10, Wherein the processing module is co-located on the system and assesses traumatic brain injury in an individual by analyzing one or more images of one or more pupils of an individual.

16. The system of claim 10, wherein the processing module is external to the system and assesses traumatic brain injury in an individual by analyzing one or more images of one or more pupils of an individual.

17. The system of claim 10, further comprising a display external to the housing.
